(19) United States
(12) Patent Application Publication
(10) Pub. No.: US 2024/0095615 A1
(43) Pub. Date: Mar. 21, 2024

(54) SYSTEMS AND METHODS FOR PROVIDING CURBSIDE INTERACTION BETWEEN PATRONS AND STAFF

(71) Applicant: H.M. Electronics, Inc., Carlsbad, CA (US)

(72) Inventor: Michael Scott Hubbard, Winter Garden, FL (US)

(21) Appl. No.: 17/353,729

(22) Filed: Jun. 21, 2021

(51) Int. Cl. G06Q 10/06; G06F 16/955; G06K 19/06; G06Q 30/06; G16H 40/20; G16H 40/67; H04L 29/08

(52) U.S. Cl. CPC G06Q 10/06311; G06F 16/9566; G06K 19/06112; G06Q 30/0633; G16H 40/20; G16H 40/67; H04L 67/146; G06K 19/06037

(57) ABSTRACT

Systems and methods for providing curbside interaction between patrons and staff members are disclosed. Exemplary implementations may: receive order information for an order placed by the patron; receive a notification from the patron upon arrival, wherein the notification may include specific location information; generate a patron-specific message; and effectuate a transfer of the patron-specific message to a wearable client device of a particular staff member.

### FIELD OF THE DISCLOSURE

The present disclosure relates to systems and methods for providing and/or facilitating curbside interaction between patrons and staff.

### BACKGROUND

Curbside services, particularly deliveries, are known. Wearable and personal communication devices are known, such as pagers, smart bands, and/or other personal communication devices. In some implementations, a pager may be an Ultra High Frequency (UHF) pager.

### SUMMARY

One aspect of the present disclosure relates to a system configured for providing curbside interaction between patrons and staff members. The system may include a local messaging transmitter, one or more hardware processors, and/or other components. The one or more hardware processors may be configured to receive order information for an order placed by the patron. The one or more hardware processors may be configured to receive a notification from the patron upon arrival. The one or more hardware processors may be configured to generate a patron-specific message. The one or more hardware processors may be configured to effectuate a transfer of the patron-specific message to a wearable client device of a particular staff member. The patron-specific message may be transferred through the local messaging transmitter.

Another aspect of the present disclosure relates to a method for providing curbside interaction between patrons and staff members. The method may include receiving order information for an order placed by the patron. The method may include receiving a notification from the patron upon arrival. The method may include generating a patron-specific message. The method may include effectuating a transfer of the patron-specific message to a wearable client device of a particular staff member. The patron-specific message may be transferred through the local messaging transmitter.

As used herein, any association (or relation, or reflection, or indication, or correspondency) involving patrons, staff members, wearable client devices, client computing platforms, servers, processors, connections, instructions, messages, notifications, transfers, user interfaces, determinations, and/or another entity or object that interacts with any part of the system and/or plays a part in the operation of the system, may be a one-to-one association, a one-to-many association, a many-to-one association, and/or a many-to-many association or “N”-to-“M” association (note that “N” and “M” may be different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured for providing curbside interaction between a patron and a particular staff member working at a place of commercial and/or medical service, in accordance with one or more implementations.

FIG. 2 illustrates a method for providing curbside interaction between a patron and a particular staff member working at a place of commercial and/or medical service, in accordance with one or more implementations.

FIG. 3A illustrates an exemplary posting at a place of commercial and/or medical service as may be used by a system configured for providing curbside interaction between a patron and a particular staff member working at the place of commercial and/or medical service, in accordance with one or more implementations.

FIG. 3B illustrates an exemplary user interface on a client computing platform as may be used by a system configured for providing curbside interaction between a patron and a particular staff member working at a place of commercial and/or medical service, in accordance with one or more implementations.

FIG. 3C illustrates an exemplary user interface on a client computing platform as may be used by a system configured for providing curbside interaction between a patron and a particular staff member working at a place of commercial and/or medical service, in accordance with one or more implementations.

FIG. 4 illustrates exemplary communications as may occur in a system configured for providing curbside interaction between a patron and a particular staff member working at a place of commercial and/or medical service, in accordance with one or more implementations.

### DETAILED DESCRIPTION

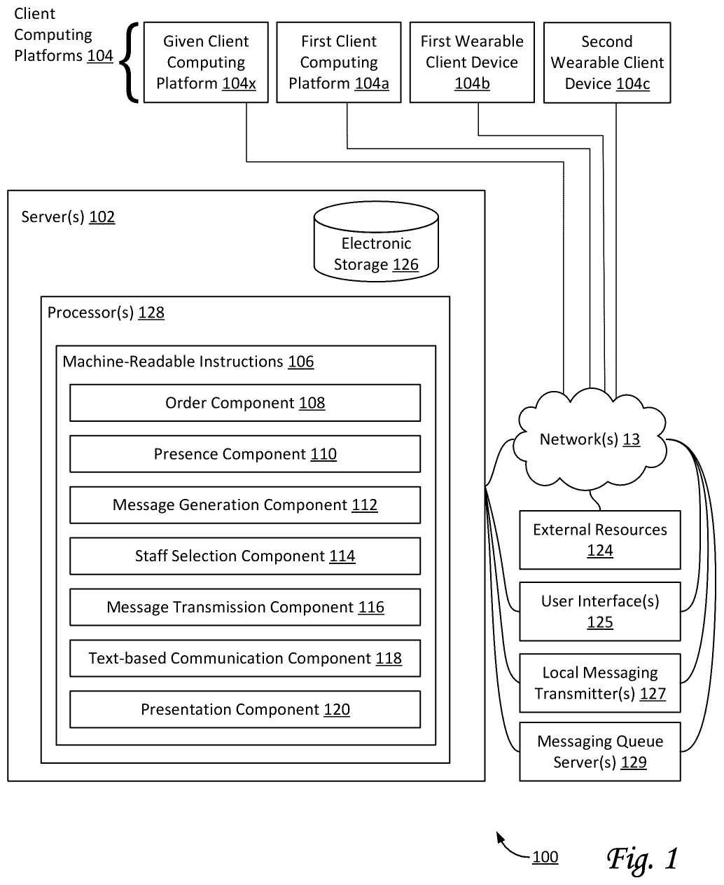

FIG. 1 illustrates a system 100 configured for providing curbside interaction between patrons and particular staff members and/or other people, in accordance with one or more implementations. In some implementations, the staff members may work at a place of commercial and/or medical service. In some implementations, the staff members may work at a place of professional service. By way of non-limiting example, staff members may work at restaurants, food service businesses, healthcare providers, pharmacies, retail stores, and/or other places of service (jointly referred to as “work places”). As used herein, the term “patrons” may refer to customers, clients, guests, and/or patients, as well as people picking up deliveries for patrons.

In some implementations, system 100 may include one or more servers 102, one or more processors 128, electronic storage 126, one or more client computing platforms 104, one or more user interfaces 125, one or more local messaging transmitters 127, one or more messaging queue servers 129, external resources 124, and/or other components. Client computing platforms 104 may include wearable client devices and/or other client computing platforms. Server(s) 102 may be configured to communicate with one or more client computing platforms 104 according to a client/server architecture, a point-to-point architecture, and/or other architectures. Client computing platform(s) 104 may be configured to communicate with other client computing platforms via server(s) 102, and/or other components. Users may access system 100 via client computing platform(s) 104. Client computing platforms 104 may include a given client computing platform 104x, a first client computing platform 104a, a first wearable client device 104b, a second wearable client device 104c, and/or other client computing platforms. Different client computing platforms 104 may be associated with different users. For example, given client computing platform 104x may be associated with a particular patron. For example, a particular wearable client device 104 may be associated with a particular staff member. In some implementations, wearable client devices may include one or more of pagers, smart bands, and/or other personal communication devices. In some implementations, a pager may be an Ultra High Frequency (UHF) pager.

In some implementations, communications between system 100 and wearable client devices 104 may be based on one or more asynchronous protocols used to transmit data to pagers. For example, in some implementations, individual wearable client devices 104 may use a communication link that is similar to or based on a Post Office Code Standardisation Advisory Group (POCSAG) communication link.

Server(s) 102 may be configured by machine-readable instructions 106. Machine-readable instructions 106 may include one or more instruction components. The instruction components may include computer program components. The instruction components may include one or more of order component 108, presence component 110, message generation component 112, staff selection component 114, message transmission component 116, text-based communication component 118, presentation component 120, and/or other instruction components.

Order component 108 may be configured to obtain and/or receive information, including but not limited to order information. Order information may be received from or on behalf of a particular patron. As used herein, “order information” may represent an order placed by a particular patron, an appointment made by a particular patron, a scheduled pickup by a particular patron, and/or other types of commercial and/or medical interactions that may include curbside interaction such as curbside service. For example, an order and/or an appointment may be placed with a place of commercial and/or medical service. For example, an order may be placed with a work place, such as a business. In some implementations, a particular patron may place an order through a given client computing platform 104x (e.g., a personal phone of the particular patron). In some implementations, order component 108 may obtain order information that is entered by a staff member on behalf of a patron.

Presence component 110 may be configured to receive information, including but not limited to notifications. In some implementations, a particular notification may be received on behalf of a patron. In some implementations, a particular notification may be received from a particular patron (or from given client computing platform 104x that is associated with a particular patron). Notifications may indicate patrons are currently at the particular place of commercial and/or medical service (and/or, through location information included in the notification, at a specific location at or near the particular work place, such as a specific designated parking spot). For example, a particular notification may indicate a particular patron is currently at a specific location of the particular work place, or has arrived at the particular work place.

In some implementations, system 100 may receive a data communication from or on behalf of a particular patron. For example, the data communication may be received from first client computing platform 104a. For example, the data communication may be a text message. In some implementations, system 100 may transfer a reply to the data communication. The reply may be another text message, and may be received by first client computing platform 104a. The reply may be presented to the particular patron on first client computing platform 104a (e.g., on a particular user interface 125 included in first client computing platform 104a). The reply may include a Uniform Resource Locator (URL), also referred to as a link. An online form and/or other web page may be located at the URL. The online form or web page may be configured such that a user can enter information. In some implementations, a particular notification received by presence component 110 may correspond to a submission of an online form (or submission of information entered in the online form and/or in another web page).

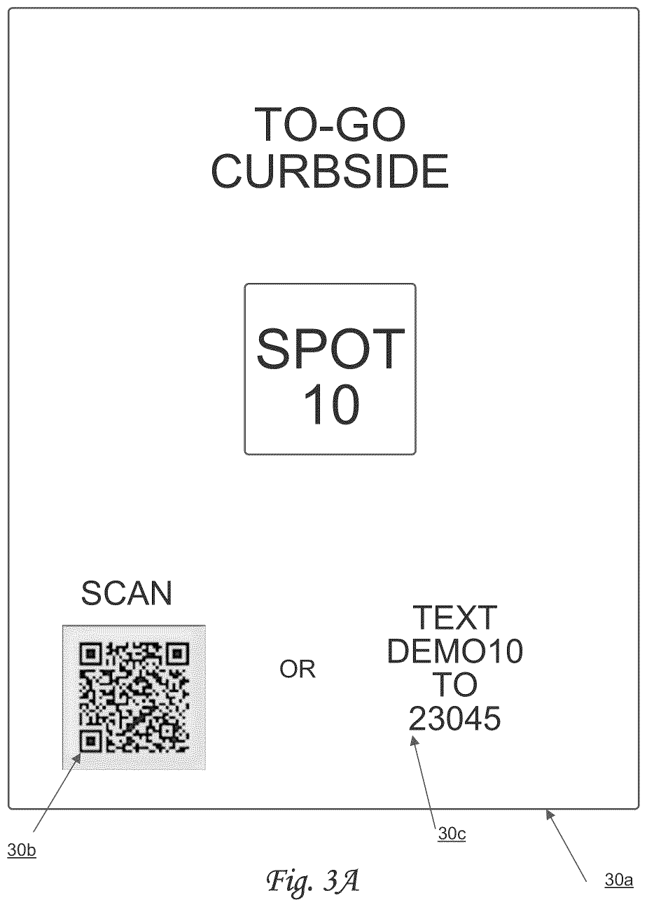

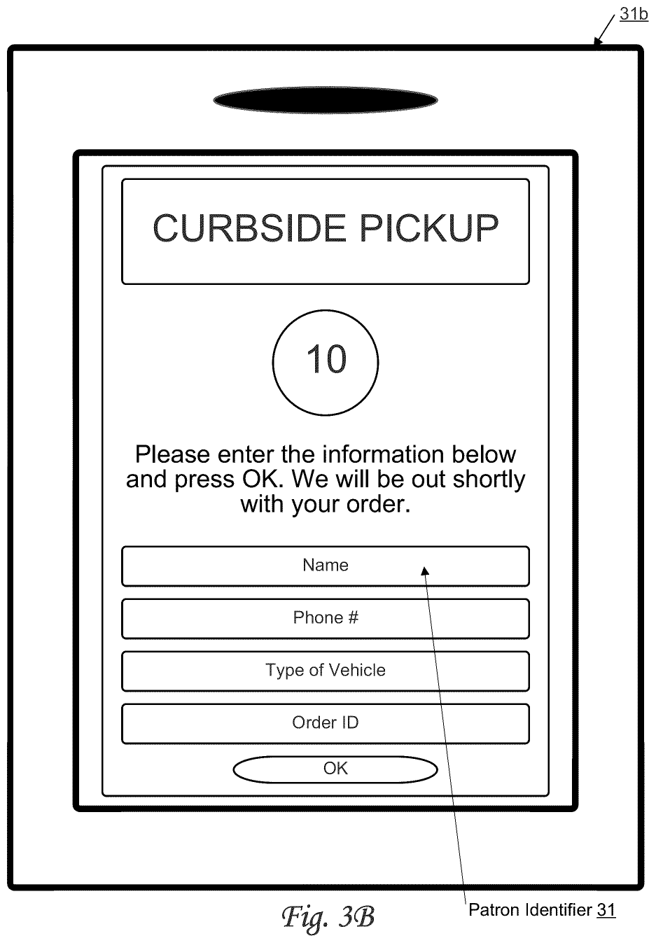

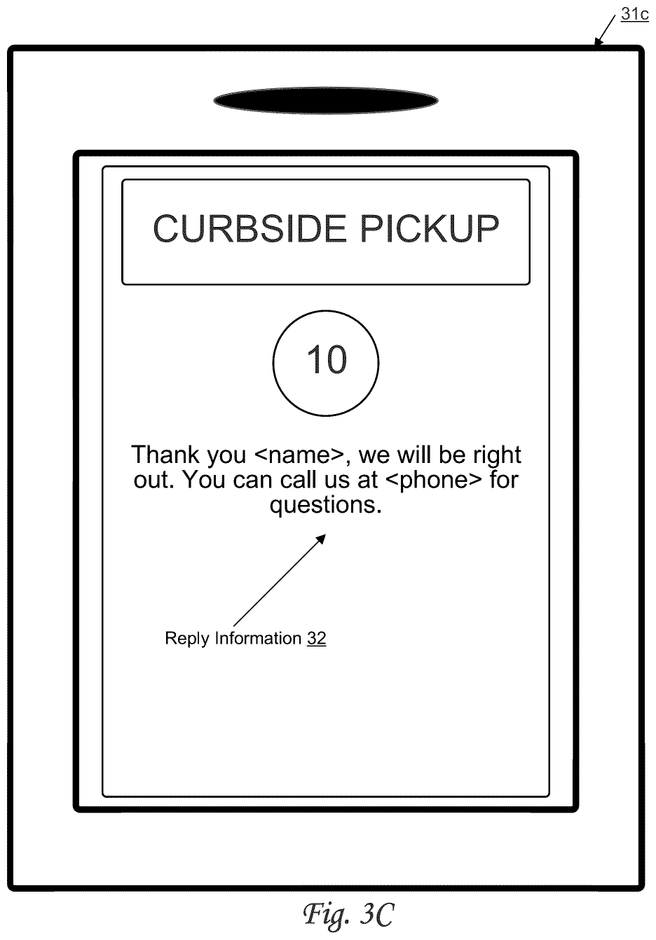

In some implementations, the URL may be accessible through a machine-readable optical label such as, by way of non-limiting example, a Quick Response (QR) code, a barcode, a two-dimensional barcode, a matrix, and/or another machine-readable optical label. For example, a QR code may be posted at the particular work place. By way of non-limiting example, FIG. 3A illustrates an exemplary posting 30a at a place of commercial and/or medical service as may be used by system 100, including a number 30c for texting and a QR code 30b. As depicted here, posting 30a is for a specific location at this work place, referred to as “Spot 10”, which may be a designated parking spot. By way of non-limiting example, FIG. 3B illustrates an exemplary user interface 31b on a client computing platform 104 (not depicted) as may be used by system 100 to present the particular URL (accessible through a QR code, or through a link in the reply to a data communication received by presence component 110). As depicted here, a particular patron may enter a patron identifier 31 that identifies the particular patron. For example, a patron identifier may be a name, a phone number, and/or other patron-specific information.

In some implementations, system 100 may transfer and/or present a reply to the notification received by presence component 110. In some implementations, a reply to the notification may be a particular data communication (e.g., a text message). In some implementations, a reply to the notification may be presented through the same URL (e.g., by refreshing and/or otherwise replacing the content at this URL upon submission of the entered information). By way of non-limiting example, FIG. 3C illustrates an exemplary user interface 31c on a client computing platform 104 (not depicted) as may be used by system 100 to present a reply, responsive to presence component 110 receiving a notification. As depicted here, the particular patron may be presented with particular reply information 32. In some implementations, reply information 32 may be specific to the particular patron, to a particular order, to a particular deliver or service, to a particular staff member, and/or any combination thereof. For example, in some cases, this information may be similar to “Hi Barney, glad you're here. Britney will bring your cheese-doodles out in 3 minutes!”. Specific information in these examples may be underlined.

Message generation component 112 may be configured to generate messages. In some implementations, a generated message may be based on (receipt of) a notification received by presence component 110. In some implementations, a generated message may be patron-specific (i.e., pertaining to a specific patron). In some implementations, a generated message may be order-specific (i.e., pertaining to a specific order). For example, the content of a generated message may be similar to “Barney is waiting at parking spot 10”, or “Barney is waiting at parking spot 10 for order 123”. Generation of messages may be performed in response to receipt of notifications by presence component 110. Contents of generated messages may be based, at least in part, on order information (e.g., obtained by order component 108). In some implementations, generated messages may be based on determinations by staff selection component 112, in addition to being based on patron-specific information.

Staff selection component 114 may be configured to determine which client computing platforms 104 (individually associated with staff members) should receive the patron-specific message generated by message generation component 112. For example, staff selection component 114 may be configured to determine a subset of client computing platforms 104 and/or corresponding staff members from a set of client computing platforms 104 and/or corresponding staff members. In some implementations, determinations by staff selection component 114 may be based on current responsibilities of one or more staff members at a particular work place (e.g., the responsibility to handle curbside deliveries). In some implementations, current responsibilities may be based on the particular role(s) of particular staff members. In some implementations, current responsibilities may be entered and/or controlled by an administrative or supervisory user of system 100. In some implementations, current responsibilities may be automatically determined based on a schedule for the staff members. In some implementations, current responsibilities may be automatically determined based on which staff members are currently working and/or checked into system 100 as currently working. For example, a restaurant may assign one or more staff members (but not all staff members, just, e.g., Britney and Brian) with the responsibility for handling take-out orders. In some implementations, a generated message may be both order-specific and specific to one or more staff members. For example, the content of a generated message may be similar to “Attention Britney, Barney is waiting at parking spot 10”, or “Attention Britney and/or Brian, Barney is waiting at parking spot 10 for order 123”. For example, one or more messages with such content may be transferred to the pagers of Britney and Brian (or some other wearable client device, as appropriate), respectively, by message transmission component 116. Upon receipt of the one or more messages, these pagers and/or other wearable client devices may present the content of the message(s) on user interfaces 125 of the pagers and/or other wearable client devices.

Message transmission component 116 may be configured to transfer messages to client computing platforms 104 (e.g., to one or more client computing platforms as determined by staff selection component 114). In some implementations, message transmission component 116 may be configured to transfer messages such that (the content of) the messages is received by client computing platforms 104 (e.g., by one or more client computing platforms as determined by staff selection component 114). In some implementations, message transmission component 116 may be configured to effectuate one or more transfers of messages such that (the content of) the messages is received by client computing platforms 104. In some implementations, a particular message may be transferred through one or more local messaging transmitters 127 (and, e.g., subsequently transferred by one or more local messaging transmitters 127 to client computing platforms 104). In some implementations, a particular message may be transferred to one or more messaging queue servers 129, subsequently received by one or more local messaging transmitters 127, and subsequently transferred by one or more local messaging transmitters 127 to client computing platforms 104.

Text-based communication component 118 may be configured to receive and/or transfer communications (particularly, data communications such as text-based communications). For example, text-based communication component 118 may receive a first data communication from first client computing platform 104a. For example, text-based communication component 118 may transfer a second data communication to first client computing platform 104a, e.g., in reply to the first data communication. The second data communication may include a URL. In some implementations, text-based communication component 118 may transfer a data communication to a patron responsive to a particular staff member taking a particular action (such as, responding to being paged).

Presentation component 120 may be configured to effectuate presentations on one or more user interfaces 125, including but not limited to user interface 125 of first client computing platform 104a. A particular presentation may be based on a message transmitted by message transmission component 116 and/or local massaging transmitter 127. In some implementations, a presentation may include aggregated information (e.g., by aggregating multiple received response messages from different interactive pagers).

Local messaging transmitter(s) 127 may be configured to receive messages, convert messages, and transfer messages. A particular local messaging transmitter may be located within communication range of a particular place of commercial and/or medical service. In some implementations, local messaging transmitter(s) 127 may receive messages that are generated by message generation component 112. In some implementations, local messaging transmitter(s) 127 may receive messages from messaging queue server(s) 129. In some implementations, local messaging transmitter(s) 127 may convert received messages into a format suitable for transfer. Local messaging transmitter(s) 127 may transfer (received and/or converted) messages to one or more client computing platforms 104 (e.g., one or more wearable client devices, as determined by staff selection component 114). In some implementations, individual local messaging transmitter(s) 127 may include one or more processors configured by machine-readable instructions to perform tasks and/or other functionality as described in this disclosure. In some implementations, individual local messaging transmitters 127 may include electronic storage configured to store information electronically. In some implementations, one or more individual local messaging transmitters 127 may be (local) paging transmitters. In some implementations, one or more individual local messaging transmitters 127 may be configured to repeatedly poll one or more messaging queue servers 129 for messages. In some implementations, one or more individual local messaging transmitters 127 may be configured to receive messages that are pushed by one or more messaging queue servers 129 to the one or more individual local messaging transmitters 127.

Messaging queue server(s) 129 may be configured to receive messages generated by message generation component 112. In some implementations, messaging queue server(s) 129 may be configured to transfer received messages to one or more client computing platforms 104 (e.g., one or more wearable client devices, as determined by staff selection component 114). In some implementations, messaging queue server(s) 129 may be configured to transfer received messages to local messaging transmitter(s) 127, which in turn transfers (the contents of) the messages to one or more client computing platforms 104 (e.g., one or more wearable client devices, as determined by staff selection component 114). In some implementations, individual messaging queue servers 129 may be configured to be repeatedly polled by individual local messaging transmitter(s) 127. In some implementations, individual messaging queue servers 129 may be configured to push messages to individual local messaging transmitter(s) 127. In some implementations, individual messaging queue server(s) 129 may include one or more processors configured by machine-readable instructions to perform tasks and/or other functionality as described in this disclosure. In some implementations, individual messaging queue server(s) 129 may include electronic storage configured to store information electronically.

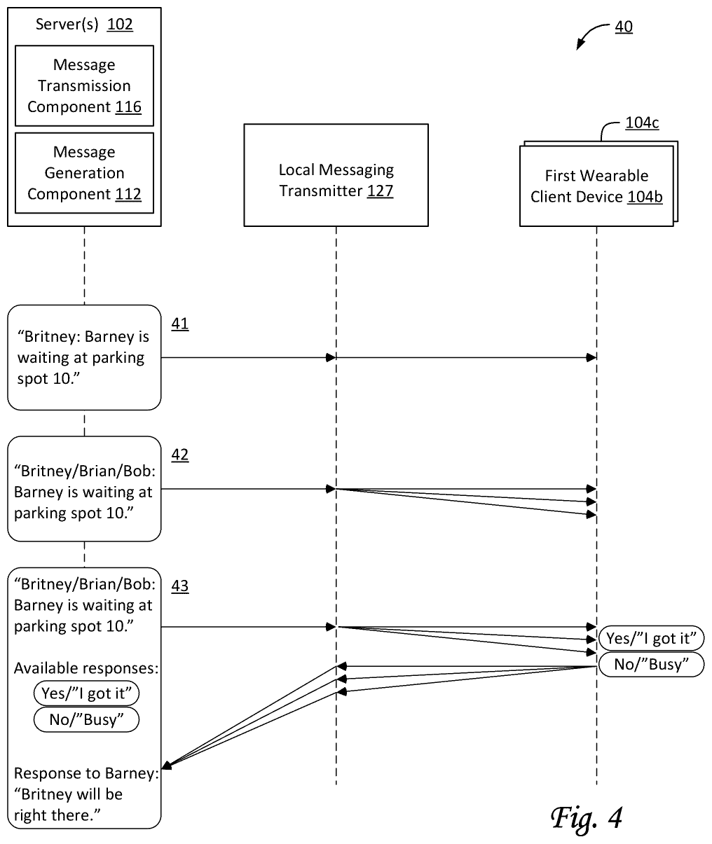

By way of non-limiting example, FIG. 4 illustrates exemplary communications as may occur in a system similar to or the same as system 100, by way of a flow diagram 40. The localized communications depicted in flow diagram 40 may occur in system 100 between message generation component 112 and/or message transmission component 116 and one or more client computing platforms 104 (e.g., one or more wearable client devices that are associated with staff members). For example, the context for flow diagram 40 may be a curbside delivery of food to Barney, at a particular restaurant, where staff members use wearable client devices. In a first communication 41, after Barney has notified the restaurant/system he is waiting, a patron-specific message is generated and transferred, through local messaging transmitter 127, with the following content: “Britney: Barney is waiting at parking spot 10.” Assume first wearable client device 104b is associated with Britney, and is configured to display the contents of this message on a user interface of first wearable client device 104b. Accordingly, first communication 41 may be a 1-on-1 communication between local messaging transmitter 127 and Britney's first wearable client device 104b. This example assumes that staff selection component 114 has determined and/or identified Britney as the appropriate recipient of this patron-specific message.

In a second communication 42, after Barney has notified the restaurant/system he is waiting, a patron-specific message is generated and transferred, through local messaging transmitter 127, with the following content: “Britney/Brian/Bob: Barney is waiting at parking spot 10.” This example assumes that staff selection component 114 has determined and/or identified Britney, Brian, and Bob as the appropriate recipients of this patron-specific message. Assume different wearable client devices are associated with each of Britney, Brian, and Bob. Assume each of these devices is configured to display the contents of this message on a user interface. Accordingly, second communication 41 may include a 1-to-many communication between local messaging transmitter 127 and a determined subset of wearable client devices, including for example first wearable client device 104b, second wearable client device 104c, and/or one or more other devices.

In a third communication 43, after Barney has notified the restaurant/system he is waiting, a patron-specific message is generated and transferred, through local messaging transmitter 127, with the following content: “Britney/Brian/Bob: Barney is waiting at parking spot 10.” This example assumes that staff selection component 114 has determined and/or identified Britney, Brian, and Bob as the appropriate recipients of this patron-specific message. Assume different wearable client devices are associated with each of Britney, Brian, and Bob. Assume each of these devices is configured to display the contents of this message on a user interface. Assume further that individual staff members who receive this message on their wearable client devices (e.g., on individual smart bands that include a button configured to provide a response, e.g., by pushing a button on their smart bands for the available responses of, by way of non-limiting example, “Yes/I got it” or “No/Busy”). More elaborate types of responses are envisioned within the scope of this disclosure. Different responses are envisioned within the scope of this disclosure. Individual responses may be used to form individual response messages. Individual response messages may be transmitted by individual wearable client devices, and received by, e.g., local messaging transmitter 127. In some implementations, local messaging transmitter 127 may transmit individual response messages (or individual messages based on individual responses) to an administrative or supervisory user (not depicted). In some implementations, local messaging transmitter 127 may be configured to aggregate received response messages into a single message and/or presentation. For example, the name of the first staff member who responds with “Yes” (here, Britney) may be used to notify Barney: “Britney will be right there.”.

In some implementations, server(s) 102, client computing platform(s) 104, and/or external resources 124 may be operatively linked via one or more electronic communication links. For example, such electronic communication links may be established, at least in part, via one or more networks 13 including but not limited to the Internet and/or other networks. It will be appreciated that this is not intended to be limiting, and that the scope of this disclosure includes implementations in which server(s) 102, client computing platform(s) 104, and/or external resources 124 may be operatively linked via some other communication media.

An individual client computing platform 104 (such as, e.g., a wearable client device) may include one or more processors configured to execute computer program components. The computer program components may be configured to enable an expert or user associated with the individual client computing platform 104 to interface with system 100 and/or external resources 124, and/or provide other functionality attributed herein to client computing platform(s) 104. By way of non-limiting example, the individual client computing platform 104 may include one or more of a desktop computer, a laptop computer, a handheld computer, a tablet computing platform, a NetBook, a phone, a pager, a gaming console, and/or other computing platforms. In some implementations, the individual client computing platform may include an individual user interface 125 (e.g., to support interaction with a user, present information, etc. etc.).

In some implementations, an individual client computing platform 104 may be a wearable client device (also referred to as wearable and personal communication device such as, e.g., a pager or smart band). Wearable client devices may be configured to generate haptic action as part of the interaction of users with the wearable client devices. For example, haptic action may include one or more vibrations. In some implementations, haptic action may be generated by a haptic engine and/or haptic component (not depicted, but commonly known from, for example, pager technology or other noted wearable technology). For example, transferring a message to an individual client computing platform 104 configured to generate specific haptic action may have the advantage that a user may be notified even if not watching a display screen, or if the particular client computing platform 104 lacks a display screen.

External resources 124 may include sources of information outside of system 100, external entities participating with system 100, and/or other resources. In some implementations, some or all of the functionality attributed herein to external resources 124 may be provided by resources included in system 100. For example, this information may be related to responsibilities among staff members for a particular work place. For example, this information may be related to work schedules for staff members for a particular work place.

Server(s) 102 may include electronic storage 126, one or more processors 128, and/or other components. Server(s) 102 may include communication lines, or ports to enable the exchange of information with one or more networks 13 (which may include the internet) and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

User interfaces 125 may be configured to facilitate interaction between users and system 100 and/or between users and client computing platforms 104. For example, user interfaces 125 may provide an interface through which users may provide information to and/or receive information from system 100. In some implementations, user interface 125 may include one or more of a display screen, touchscreen, monitor, a keyboard, buttons, switches, knobs, levers, mouse, speakers, headphones, microphones, sensors to capture voice commands, sensors to capture eye movement and/or body movement, sensors to capture hand and/or finger gestures, and/or other user interface devices configured to receive and/or convey user input. In some implementations, one or more user interfaces 125 may be included in one or more client computing platforms 104. In some implementations, one or more user interfaces 125 may be included in system 100.

Electronic storage 126 may comprise non-transitory storage media that electronically stores information. The electronic storage media of electronic storage 126 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 126 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 126 may include one or more virtual storage resources (e.g., cloud storage, a virtual private network, and/or other virtual storage resources). Electronic storage 126 may store software algorithms, information determined by processor(s) 128, information received from server(s) 102, information received from client computing platform(s) 104, and/or other information that enables server(s) 102 to function as described herein. For example, stored information may be related to responsibilities among staff members for a particular work place. For example, stored information may be related to work schedules for staff members for a particular work place.

Processor(s) 128 may be configured to provide information processing capabilities in server(s) 102. As such, processor(s) 128 may include one or more of a digital processor, an analog processor, a digital circuit designed to process information, an analog circuit designed to process information, a state machine, and/or other mechanisms for electronically processing information. Although processor(s) 128 is shown in FIG. 1 as a single entity, this is for illustrative purposes only. In some implementations, processor(s) 128 may include a plurality of processing units. These processing units may be physically located within the same device, or processor(s) 128 may represent processing functionality of a plurality of devices operating in coordination. Processor(s) 128 may be configured to execute components 108, 110, 112, 114, 116, 118, and/or 120, and/or other components. Processor(s) 128 may be configured to execute components 108, 110, 112, 114, 116, 118, and/or 120, and/or other components by software; hardware; firmware; some combination of software, hardware, and/or firmware; and/or other mechanisms for configuring processing capabilities on processor(s) 128. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, 114, 116, 118, and/or 120 are illustrated in FIG. 1 as being implemented within a single processing unit, in implementations in which processor(s) 128 includes multiple processing units, one or more of components 108, 110, 112, 114, 116, 118, and/or 120 may be implemented remotely from the other components. The description of the functionality provided by the different components 108, 110, 112, 114, 116, 118, and/or 120 described below is for illustrative purposes, and is not intended to be limiting, as any of components 108, 110, 112, 114, 116, 118, and/or 120 may provide more or less functionality than is described. For example, one or more of components 108, 110, 112, 114, 116, 118, and/or 120 may be eliminated, and some or all of its functionality may be provided by other ones of components 108, 110, 112, 114, 116, 118, and/or 120. As another example, processor(s) 128 may be configured to execute one or more additional components that may perform some or all of the functionality attributed below to one of components 108, 110, 112, 114, 116, 118, and/or 120.

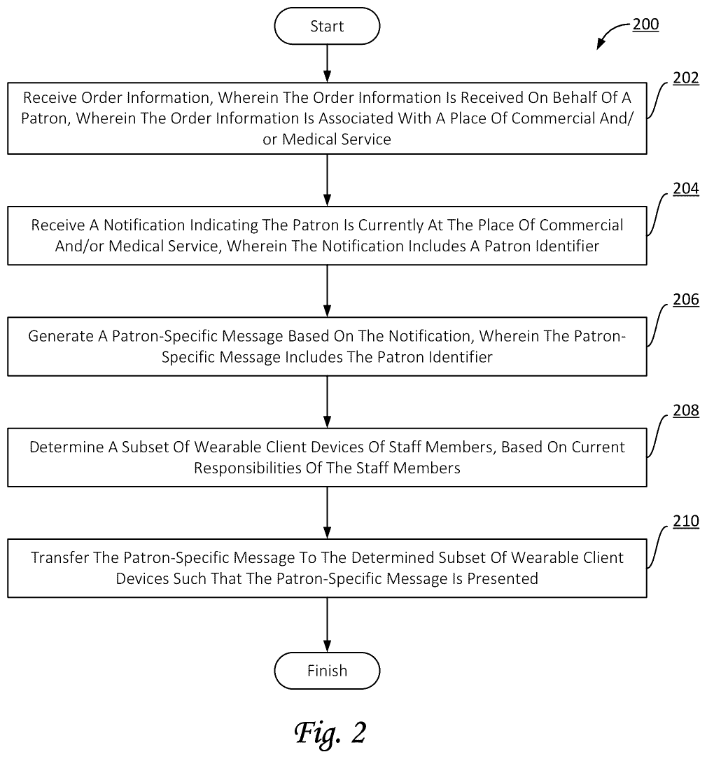

FIG. 2 illustrates a method 200 for providing curbside interaction between a patron and a particular staff member working at a place of commercial and/or medical service, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, order information is received. The order information may represent an order placed by the patron, an appointment made by the patron, or a scheduled pickup by the patron, through a given client computing platform, on behalf of the patron, wherein the order information is associated with the place of commercial and/or medical service. In some embodiments, operation 202 is performed by an order component the same as or similar to order component 108 (shown in FIG. 1 and described herein).

At an operation 204, a notification is received, on behalf of the patron. The notification indicates the patron is currently at the place of commercial and/or medical service. The notification may indicate a specific location of the patron, which may be referred to as a patron location. The notification is received from a first client computing platform associated with the patron. The notification includes a patron identifier that identifies the patron. In some embodiments, operation 204 is performed by a presence component the same as or similar to presence component 110 (shown in FIG. 1 and described herein).

At an operation 206, a patron-specific message is generated based on the notification. The patron-specific message includes the patron identifier and may include the patron location. In some embodiments, operation 206 is performed by a message generation component the same as or similar to message generation component 112 (shown in FIG. 1 and described herein).

At an operation 208, a subset is determined of one or more wearable client devices of one or more staff members at the place of commercial and/or medical service. The subset includes a particular wearable client device of the particular staff member. The determination is based on current responsibilities of the one or more staff members. In some embodiments, operation 208 is performed by a staff selection component the same as or similar to staff selection component 114 (shown in FIG. 1 and described herein).

At an operation 210, a transfer of the patron-specific message is effectuated, through a local messaging transmitter, to the determined subset of the one or more wearable client devices of the one or more staff members at the place of commercial and/or medical service, including the particular wearable client device such that the patron-specific message is presented on a user interface that is included in the particular wearable client device. In some embodiments, operation 210 is performed by a message transmission component the same as or similar to message transmission component 116 (shown in FIG. 1 and described herein).

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured for generating and effectuating transfer of patron-specific messages that facilitate providing curbside interaction between a patron and a particular staff member working at a place of commercial and/or medical service, the system comprising:
a local messaging transmitter including one or more processors configured by machine-readable instructions to receive messages and transfer the messages to one or more wearable client devices of one or more staff members at the place of commercial and/or medical service, wherein the local messaging transmitter is located within communication range of the place of commercial and/or medical service; and
one or more hardware processors configured by machine-readable instructions to:
receive order information, wherein the order information represents an order placed by the patron, through a given client computing platform, with the place of commercial and/or medical service;
receive a notification, on behalf of the patron, wherein the notification indicates the patron is currently at the place of commercial and/or medical service, wherein the notification is received from a first client computing platform associated with the patron, wherein the notification includes a patron identifier that identifies the patron;
generate a patron-specific message based on the notification, wherein the patron-specific message includes the patron identifier;
automatically determine a subset of the one or more wearable client devices of the one or more staff members at the place of commercial and/or medical service, wherein the subset includes a particular wearable client device of the particular staff member, wherein the determination is based on current responsibilities of the one or more staff members; and
effectuate a transfer of the patron-specific message, through the local messaging transmitter, to the determined subset of the one or more wearable client devices of the one or more staff members at the place of commercial and/or medical service, including the particular wearable client device such that the patron-specific message is presented on a user interface that is included in the particular wearable client device.

2. The system of claim 1, wherein the one or more hardware processors are further configured to:
receive a data communication on behalf of the patron, wherein the data communication is received from the first client computing platform;
transfer a reply to the data communication, wherein the reply includes a Uniform Resource Locator (URL);
wherein the notification corresponds to a submission of an online form, wherein the online form is located at the Uniform Resource Locator (URL) included in the reply.

3. The system of claim 1, wherein the notification corresponds to a submission of an online form, wherein the online form is located at a Uniform Resource Locator (URL) that is accessible through a machine-readable optical label displayed at the place of commercial and/or medical service.

4. The system of claim 1, wherein the notification further includes location information of a particular location of the patron at the place of commercial and/or medical service.

5. The system of claim 1, wherein the determination of the subset of the one or more wearable client devices is performed automatically based on a current schedule that includes which of the one or more staff members is currently working at the place of commercial and/or medical service.

6. The system of claim 1, wherein the one or more client devices of the one or more staff members are configured to generate haptic action in response to receiving the patron-specific message.

7. The system of claim 1, wherein effectuating the transfer includes:
transferring the patron-specific message to a messaging queue server;
wherein the local messaging transmitter is configured to receive the patron-specific message from the messaging queue server, and wherein the local messaging transmitter subsequently transfers the patron-specific message to the determined subset of the one or more wearable client devices, including the particular wearable client device.

8. The system of claim 7, wherein the local messaging transmitter is configured to repeatedly poll the messaging queue server prior to receiving the patron-specific message.

9. The system of claim 7, wherein the messaging queue server is configured to push the patron-specific message to the local messaging transmitter.

10. The system of claim 8, wherein the local messaging transmitter is a local paging transmitter, wherein the one or more wearable client devices include one or more of Ultra High Frequency (UHF) pagers and smart bands capable of haptic action, wherein the messaging queue server is a paging queue server, and wherein the patron-specific message is transferred to the one or more wearable client devices through paging and by using the haptic action.

11. A method for generating and effectuating transfer of patron-specific messages that facilitate providing curbside interaction between a patron and a particular staff member working at a place of commercial and/or medical service, the method comprising:
receiving order information, wherein the order information represents an order placed on behalf of the patron, through a given client computing platform, wherein the order information is associated with the place of commercial and/or medical service;
receiving a notification, on behalf of the patron, wherein the notification indicates the patron is currently at the place of commercial and/or medical service, wherein the notification is received from a first client computing platform associated with the patron, wherein the notification includes a patron identifier that identifies the patron;
generating a patron-specific message based on the notification, wherein the patron-specific message includes the patron identifier;
automatically determining a subset of one or more wearable client devices of one or more staff members at the place of commercial and/or medical service, wherein the subset includes a particular wearable client device of the particular staff member, wherein the determination is based on current responsibilities of the one or more staff members; and
effectuating a transfer of the patron-specific message, through a local messaging transmitter, to the determined subset of the one or more wearable client devices of the one or more staff members at the place of commercial and/or medical service, including the particular wearable client device such that the patron-specific message is presented on a user interface that is included in the particular wearable client device.

12. The method of claim 11, further comprising:
receiving a data communication on behalf of the patron, wherein the data communication is received from the first client computing platform; and
transferring a reply to the data communication, wherein the reply includes a Uniform Resource Locator (URL);
wherein the notification corresponds to a submission of an online form, wherein the online form is located at the Uniform Resource Locator (URL) included in the reply.

13. The method of claim 11, wherein the notification corresponds to a submission of an online form, wherein the online form is located at a Uniform Resource Locator (URL) that is accessible through a machine-readable optical label displayed at the place of commercial and/or medical service.

14. The method of claim 11, wherein the notification further includes location information of a particular location of the patron at the place of commercial and/or medical service.

15. The method of claim 11, wherein determining the subset of the one or more wearable client devices is performed automatically based on a current schedule that includes which of the one or more staff members is currently working at the place of commercial and/or medical service.

16. The method of claim 11, wherein the one or more client devices of the one or more staff members generate haptic action in response to receiving the patron-specific message.

17. The method of claim 11, wherein effectuating the transfer includes:
transferring the patron-specific message to a messaging queue server;
wherein the local messaging transmitter receives the patron-specific message from the messaging queue server, and wherein the local messaging transmitter subsequently transfers the patron-specific message to the determined subset of the one or more wearable client devices, including the particular wearable client device.

18. The method of claim 17, wherein the local messaging transmitter repeatedly polls the messaging queue server prior to receiving the patron-specific message.

19. The method of claim 17, wherein the messaging queue server pushes the patron-specific message to the local messaging transmitter.

20. The method of claim 18, wherein the local messaging transmitter is a local paging transmitter, wherein the one or more wearable client devices include one or more of Ultra High Frequency (UHF) pagers and smart bands capable of haptic action, wherein the messaging queue server is a paging queue server, and wherein the patron-specific message is transferred to the one or more wearable client devices through paging and by using the haptic action.
